(19) United States
(12) Patent Application Publication
(10) Pub. No.: US 2025/0042888 A1
(43) Pub. Date: Feb. 6, 2025

(54) A PROCESS FOR THE PREPARATION OF INDOLMYCIN AND DERIVATIVES THEREOF

(71) Applicant: COUNCIL OF SCIENTIFIC AND INDUSTRIAL RESEARCH AN INDIAN REGISTERED BODY INCORPORATED UNDER THE REGN., New Delhi, Delhi (IN)

(72) Inventors: Syed GULAM DASTAGER, Pune Maharashtra (IN); Madhukar SHYAM SAID, Pune Maharashtra (IN); Abujanaid KHAN, Maharashtra Pune (IN); Amol ARVIND KULKARNI, Maharashtra Pune (IN)

(73) Assignee: COUNCIL OF SCIENTIFIC AND INDUSTRIAL RESEARCH AN INDIAN REGISTERED BODY INCORPORATED UNDER THE REGN., New Delhi, Delhi (IN)

(21) Appl. No.: 18/710,553

(22) PCT Filed: Nov. 24, 2022
(86) PCT No.: PCT/IN2022/051026

§ 371 Date: May 15, 2024

(30) Foreign Application Priority Data
Nov. 26, 2021 (IN) 202111055049

(51) Int. Cl. C07D 413/06

(52) U.S. Cl. CPC C07D 413/06

(57) ABSTRACT

The present disclosure relates to a new, simple, two step synthesis of Indolmycin and its derivatives of formula (I), in 4-5 hours. Formula (I) wherein said process comprising the step of: 10 (a) reacting indole derivative with methyl magnesium bromide in a suitable solvent at a temperature in the range of 0-30° C. followed by adding ethyl epoxy butanone in a suitable solvent at −20° C. for a period of 1-2 hrs and maintaining the obtained reaction mixture for 1 hr at −20° C. to obtain a compound; and (b) passing the compound obtained at step a), base and N-methylated quinidine 15 through solid-solid continuous flow reactor for 1 minute to obtain the compound of formula (I).

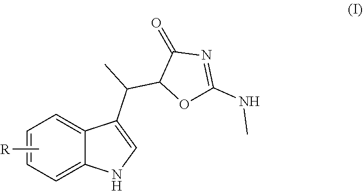

### FIELD OF THE INVENTION

The present disclosure relates to a process for the preparation of Indolmycin and its derivatives of formula (I). More particularly, the present disclosure relates to a new, simple, two step synthesis of Indolmycin and its derivatives of formula (I) in 4-5 hours.

### BACKGROUND AND PRIOR ART OF THE INVENTION

Indolmycin is a member of the class of 1,3-oxazoles that is 1,3-oxazol-4 (5H)-one which is substituted at the 2 and 5-pro-S positions by methylamino and [(IR)-1-(1H-indol-3-yl)ethyl] groups, respectively. It has a role as an antimicrobial agent, an EC 6.1.1.2 (tryptophan—tRNA ligase) inhibitor, an antibacterial agent and a bacterial metabolite. Indolmycin is a highly selective antibiotic, which acts as a tryptophan anti-metabolite. Recent research has shown that indolmycin is active against Mycobacteria and H. pylori and can stimulate transcription in Escherichia coli.

Several prior arts report synthesis of Indolmycin. An article entitled “Total synthesis of antibiotic indolmycin and its stereoisomers” by M. N. Preobrazhenskaya et al. and published in the journal “Tetrahedron, Volume 24, Issue 19, 1968, Pages 6131-6143” reports total synthesis Indolmycin. The process involves the optical resolution of a racemic mixture of indolebutyric acid followed by cyclisation with N, N′-dimethylguanidine.

An article entitled “Development of a stereoselective practical synthetic route to indolmycin, a candidate anti-H. pylori agent” by A Hasuoka et al. and published in the journal “Chem Pharm Bull (Tokyo), 2001 December; 49 (12): 1604-8 reports synthesis of indolmycin. The process involves the condensation of chiral epoxide with indole in the presence of methyl magnesium bromide to give indolyl butyric ester which on basic hydrolysis and crystallization gives corresponding butyric acid. The esterification of obtained acid with ethanol and HCl provides pure indolyl butyric ester, which on cyclisation with guanidine in the presence of potassium t-butoxide in tert-butanol gives corresponding oxazolone. Further methylation of the amino group with methyl amine gives indolmycin.

Prior arts suffer drawbacks like lengthy synthetic routes involving 3-4 steps, which take a long time of 6-7 days, very poor yield of <70%, requirement of solvents at each step, and only batch method.

Therefore, there is a need in the art to develop a synthetic route giving good yield in shorter time, with few steps and at cheaper cost.

### OBJECTS OF THE INVENTION

The main objective of the present disclosure is to provide a new, simple continuous flow two-step process for the synthesis of Indolmycin and its derivatives compound of formula (I).

### SUMMARY OF THE INVENTION

Accordingly, to accomplish the objectives, the present disclosure provides a new, simple, continuous flow, two-step process for the synthesis of Indolmycin compound and its derivatives of formula (I) with more than 70% yield.

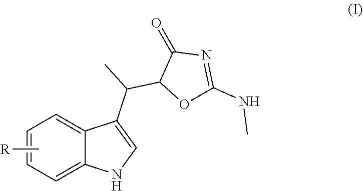

wherein,
R is selected from the group comprising of hydrogen, C₁-C₁₀ alkyl, C₁-C₁₀ aryl, alkyl aryl, haloalkyl, hydroxy alkyl, alkoxy, hydroxy, halo, cyano, heteroaryl, heteroalkyl, alkyl heteroaryl, substituted or unsubstituted C₁-C₁₀ aryl/alkyl, C₂-C₁₀ alkenyl, alkenyl aryl, alkenyl heteroaryl, C₂-C₁₀ alkynyl, alkynyl aryl, alkynyl heteroaryl, C₃-C₁₀ cycloalkyl, heterocycloalkyl, alkyl cycloalkyl, alkyl heterocycloalkyl, alkyl carboxy, acyl, alkyl acyl, alkyl acyloxy, alkyl alkoxy, alkyl carbonyl, aryl carbonyl, alkoxycarbonyl, alkyl alkoxycarbonyl, aminocarbonyl, alkyl aminocarbonyl, alkyl acylamino, alkyl ureido, amino, alkyl amino, sulfonyloxy, alkyl sulfonyloxy, sulfonyl, alkyl sulfonyl, sulfinyl, alkyl sulfinyl, alkyl sulfanyl and alkyl sulfonylamino.

In an embodiment, the present disclosure provides a continuous flow process for the preparation of Indolmycin and its derivatives compound of formula (I) (wherein R is as defined above),

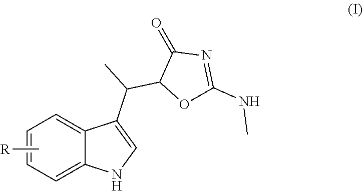

wherein said process comprises the steps of:
a) reacting indole derivative compound (2)

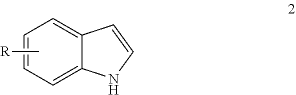

with methyl magnesium bromide in a suitable solvent at a temperature in the range of 0-20° C., followed by adding ethyl epoxybutanone of compound (1)

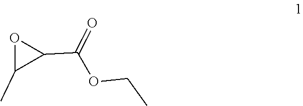

in a suitable solvent at −20° C. for a period of 1-2 hrs. and maintaining reaction mixture for 1 hr at −20° C. to afford the compound of formula (3);

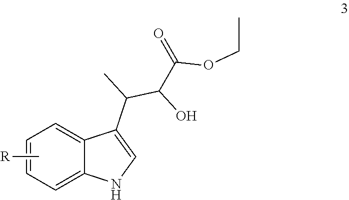

b) passing the compound of formula (3) obtained at step a), tertiary butoxide and N methylated quinidine compound (4)

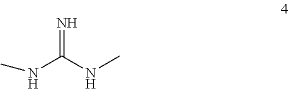

through solid-solid continuous flow reactor for 1 minute to afford the compound of formula (I).

In an embodiment of the present disclosure, the R is hydrogen or C₁-C₁₀ alkyl.

In an embodiment, the process of the present disclosure is very simple, continuous flow involving only two steps.

In another embodiment of the present disclosure, the process of preparing Indolmycin completes in only 2-5 hours compared to lengthy processes of 6-7 days reported in prior arts/literature known processes.

In another aspect of the present disclosure, the solvent used in the process is selected from tert-butyl alcohol, dichloromethane, ethyl acetate and water.

Various objects, features, aspects and advantages of the inventive subject matter will become more apparent from the following detailed description of preferred embodiment.

### BRIEF DESCRIPTION OF THE DRAWINGS

The following drawing form a part of the present specification and is included to further illustrate aspects of the present disclosure. The disclosure may be better understood by reference to the drawing in combination with the detailed description of the specific embodiments presented herein.

FIG. 1 depicts the flow reactor used to synthesize the compound of formula (I): (1) solid dosing-1 (2) solid dosing-2 (3) motor (4) jacket outlet (5) Jacket (6) screw conveyor (7) jacket inlet and (8) product, in accordance with an embodiment of the present disclosure.

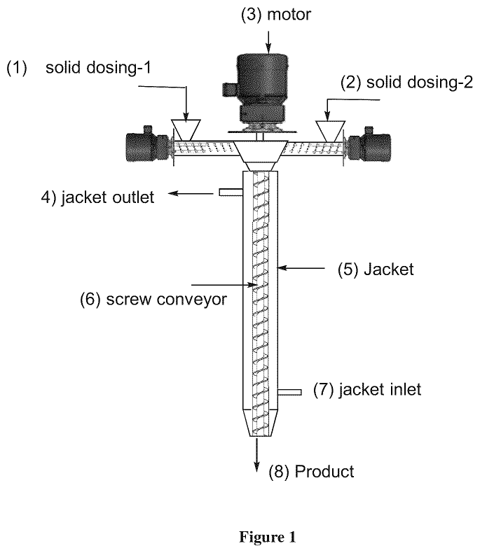

### DETAILED DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

It is to be understood that the figures and descriptions of the present disclosure have been simplified to illustrate elements that are relevant for a clear understanding of the disclosure. The detailed description will be provided herein below with reference to the attached drawing.

### Definitions

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”. Throughout this specification, unless the context requires otherwise the word “comprise”, and variations such as “comprises” and “comprising”, will be understood to imply the inclusion of a stated element or step or group of element or steps but not the exclusion of any other element or step or group of element or steps. In the description, term “(C₁-C₁₀) alkyl” refer to the non-ring filling of the linear chain or branched chain with 1 to 10 carbon atom(s). Hydrocarbon, term “(C₁-C₁₀) alkoxy” refer to the non-cyclic hydrocarbon of the linear chain or branched chain with more than one ether and 1 to 10 carbon atom(s).

The alicyclic hydrocarbon groups in the definition of the hydrocarbon group may be saturated or unsaturated, and include cycloalkyl group, cycloalkenyl group, cycloalkadienyl group, etc. The cycloalkyl groups include ones having 3 to 9 carbon atoms Such as cyclopropyl, cyclobutyl, cyclopentyl, cyclohexyl, cycloheptyl, cyclooctyl, cyclononyl, etc. Among them, Cycloalkyl groups Such as cyclopropyl, cyclobutyl, cyclopentyl, cyclohexyl, etc. are preferable. The cycloalkenyl groups include C-cycloalkenyl Such as 2-cyclopenten-1-yl, 3-cyclopentene-1-yl, 2-cyclohexen-1-yl, 3-cyclohexen-1-yl, 1-cyclobutene-1-yl, 1-cyclopentene-1-yl, etc. The cycloalkadienyl groups include Cyclopentadienyl Such as 2,4-cyclopentadiene-1-yl, 2,4-cyclohexadiene-1-yl, 2,5-cyclohexadiene-1-yl, etc.

Furthermore, unless stated otherwise, the alkyl group can be unsubstituted or substituted with one or more substituents, for example, from one to four substituents, independently selected from the group consisting of halogen, hydroxy, cyano, nitro and amino. Examples of substituted alkyl include, but are not limited to hydroxymethyl, 2-chlorobutyl, trifluoromethyl and aminoethyl.

The term, “halogen” as used herein refers to chlorine, fluorine, bromine or iodine atoms.

Ratios, concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a temperature in the range of −20° C. to 20° C. should be interpreted to include not only the explicitly recited limits of −20° C. to 20° C. but also to include sub-ranges, such as −1° C.-10° C., and so forth, as well as individual amounts, within the specified ranges, such as −5.2° C., 0.5° C., 10.5° C., 19.2° C. and so on.

Prior arts in the field of preparation of the compound of formula (I) Indolmycin suffer drawbacks like overlong synthetic routes involving 3-4 steps, which take a long time of 6-7 days, very poor yield of <70%, requirement of solvents at each step, and only batch method. Therefore, there is an unmet need in the field to develop a synthetic route to produce Indolmycin with good yield in short time, few steps and cheap cost.

Accordingly, to accomplish the objectives, the present disclosure provides a new, continuous flow, simple two-step process for the synthesis of Indolmycin and its derivatives compound of formula (I), in more than 70% yield.

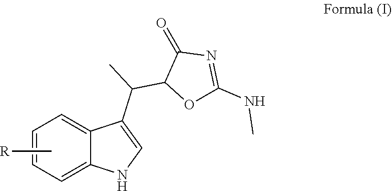

wherein,
R is selected from the group comprising of hydrogen, C₁-C₁₀ alkyl, C₁-C₁₀ aryl, alkyl aryl, haloalkyl, alkyl hydroxy, alkoxy, hydroxy, halo, cyano, heteroaryl, heteroalkyl, alkyl heteroaryl, substituted or unsubstituted C₁-C₁₀ aryl/alkyl, C₂-C₁₀ alkenyl, alkenyl aryl, alkenyl heteroaryl, C₂-C₁₀ alkynyl, alkynyl aryl, alkynyl heteroaryl, C₃-C₁₀ cycloalkyl, heterocyclic alkyl, alkyl cycloalkyl, alkyl heterocycloalkyl, alkyl carboxy, acyl, alkyl acyl, alkyl acyloxy, alkyl alkoxy, alkyl carbonyl, aryl carbonyl, alkoxycarbonyl, alkyl alkoxycarbonyl, aminocarbonyl, alkyl aminocarbonyl, alkyl acylamino, alkyl ureido, amino, alkyl amino, sulfonyloxy, alkyl sulfonyloxy, sulfonyl, alkyl sulfonyl, sulfinyl, alkyl sulfinyl, alkyl sulfanyl and alkyl sulfonyl amino;
wherein said process comprising the step of:
a) reacting indole derivative compound (2)

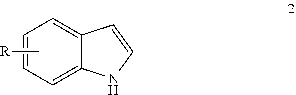

with methyl magnesium bromide in a suitable solvent at a temperature in the range of 0-30° C., followed by adding ethyl epoxy butanone compound (1)

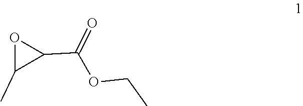

in a suitable solvent at −20° C. for a period of 1-2 hrs and maintaining the obtained reaction mixture for 1 hr at −20° C. to obtain the compound of formula (3); and

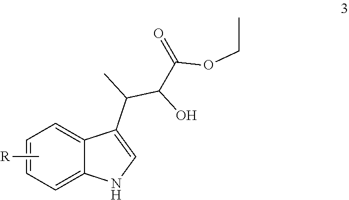

b) passing the compound of formula (3) obtained at step a), base and N-methylated quinidine (4) through solid-solid continuous flow reactor for 1 minute to obtain the compound of formula (I);

In another embodiment of the present disclosure, there is provided a process as disclosed herein, wherein the R is hydrogen or C₁-C₁₀ alkyl.

In an embodiment of the present disclosure, there is provided a continuous flow process as disclosed herein, wherein the base is selected from tertiary butoxide, sodium tert butoxide, triethylamine, diisopropylethylamine, pyridine, lutidine, procaine, 2-phenyl ethyl benzylamine, dibenzylethylenediamine, ethanolamine, diethanolamine, tris hydroxymethyl aminomethane, polyhydroxy alkylamine, or N-methyl glucosamine.

In an embodiment of the present disclosure, there is provided a process for the preparation of Indolmycin, and its derivatives compound of formula (I) is depicted below in general scheme-1:

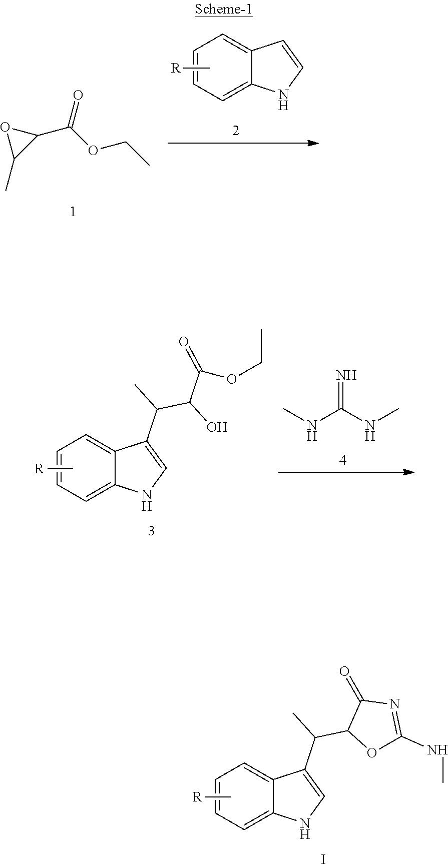

wherein,
R is selected from the group comprising of hydrogen, C₁-C₁₀ alkyl, C₁-C₁₀ aryl, alkyl aryl, haloalkyl, alkyl hydroxy, alkoxy, hydroxy, halo, cyano, heteroaryl, heteroalkyl, alkyl heteroaryl, substituted or unsubstituted C₁-C₁₀ aryl/alkyl, C₂-C₁₀ alkenyl, alkenyl aryl, alkenyl heteroaryl, C₂-C₁₀ alkynyl, alkynyl aryl, alkynyl heteroaryl, C₃-C₁₀ cycloalkyl, heterocycloalkyl, alkyl cycloalkyl, alkyl heterocycloalkyl, alkyl carboxy, acyl, alkyl acyl, alkyl acyloxy, alkyl alkoxy, alkyl carbonyl, aryl carbonyl, alkoxycarbonyl, alkyl alkoxycarbonyl, aminocarbonyl, alkyl aminocarbonyl, alkyl acylamino, alkyl ureido, amino, alkyl amino, sulfonyloxy, alkyl sulfonyloxy, sulfonyl, alkyl sulfonyl, sulfinyl, alkyl sulfinyl, alkyl sulfonyl and alkyl sulfonyl amino. In another embodiment of the present disclosure, there is provided a process as disclosed herein, wherein the R is hydrogen or C₁-C₁₀ alkyl.

In an embodiment, the present disclosure provides a continuous flow process for the preparation of Indolmycin and its derivatives compound of formula (I), wherein said process comprises the steps of:

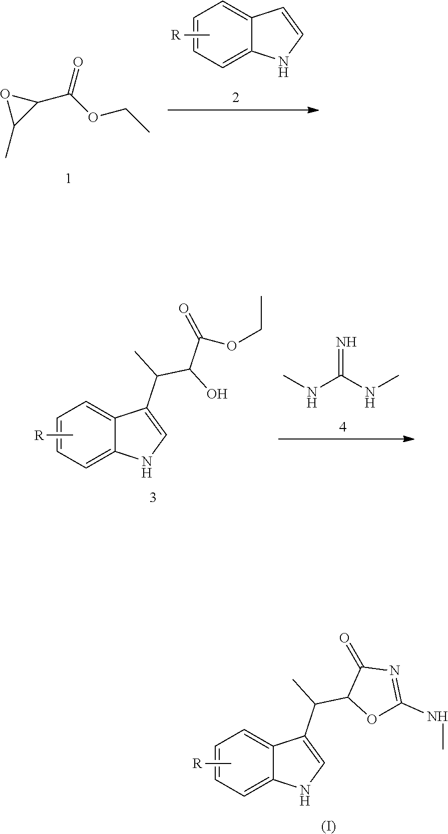

a) reacting indole derivative compound (2) with methyl magnesium bromide in a suitable solvent at a temperature in the range of 0-20° C., followed by adding ethyl epoxybutanone-1 in a suitable solvent at −20° C. for a period of 1-2 hrs and maintaining the obtained reaction mixture for 1 hr at −20° C. to afford the compound of formula 3; and
b) passing the compound of formula (3) obtained at step a), tertiary butoxide and N methylated quinidine (4) through solid-solid continuous flow reactor for 1 minute to afford compound of formula (I) (wherein R is as defined above).

In an embodiment of the present disclosure, there is provided a continuous flow process for the preparation of Indolmycin comprising the steps of:
a) a) reacting indole with methyl magnesium bromide in a suitable solvent at a temperature in the range of 0-20° C., followed by adding ethyl epoxybutanone of compound (1)

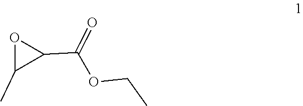

in a suitable solvent at −20° C. for a period of 1-2 hours and maintaining reaction mixture for 1 hour at −20° C. to afford the compound (3a);

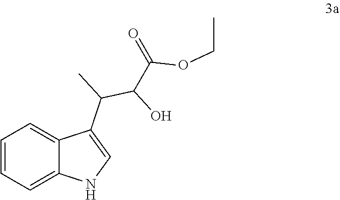 [SMILES: CCOC(=O)C(O)C(C)c1c[nH]c2ccccc12]

b) passing the compound (3a) obtained at step (a), tertiary butoxide and N methylated quinidine (4)

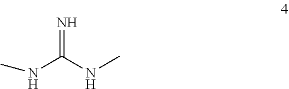

through solid-solid continuous flow reactor for 1 minute to obtain Indolmycin. In another aspect of the present disclosure, the process is very simple, continuous flow, and cost-effective involving only two steps. The process completes in only 2-5 hours compared to prior art lengthy processes of 6-7 days.

In another embodiment of the present disclosure, there is provided a continuous flow process for the preparation of Indolmycin and its derivatives as disclosed herein, wherein the solvent used in the process is selected from the group consisting of tert-butyl alcohol, dichloromethane, ethyl acetate and water, methanol, ethanol, acetone, methyl ethyl ketone, acetonitrile, propionitrile, formamide, N,N-dimethylformamide, dioxane, tetrahydrofuran, dimethyl and sulfoxide, sulfolane hexamethylphosphoramide. These solvents can be used alone or in combination thereof. Preferably, the solvent is dichloromethane, ethyl acetate, tetrahydrofuran, or combination thereof.

In an embodiment of the present disclosure, there is provided a continuous flow process for the preparation of Indolmycin, and its derivatives as disclosed herein, wherein the step a) of the process further comprises acidifying the reaction mixture using acids to obtain the compound of formula (3). In another embodiment of the present disclosure, the acid used in the process is selected from the group consisting of inorganic acids such as hydrochloric acid, sulfuric acid, hydrogen bromide, hydrogen iodide, phosphonic acid, sulfamic acid, etc. and organic acids Such as acetic acid, lactic acid, Succinic acid, maleic acid, tartaric acid, citric acid, gluconic acid, ascorbic acid, benzoic acid, methane sulfonic acid, p-toluene sulfonic acid, cinnamic acid, fumaric acid, etc. Preferably, the acid is hydrochloric acid, sulfuric acid, hydrogen bromide, hydrogen iodide, phosphonic acid, or sulfamic acid. In another embodiment of the present disclosure, the acid used in the process is preferably selected from inorganic acids such as hydrochloric acid.

In yet another an embodiment of the present disclosure, there is provided a continuous flow process for the preparation of Indolmycin, and its derivatives as disclosed herein, wherein the reaction temperature is in the range of −20° C. to 30° C.

In an embodiment of the present disclosure, there is provided a comparative study of the present disclosure with prior art which indicates that the process of the present disclosure is highly economical for commercialization giving high yield of at least 70% with less steps and lesser time. Comparative study with prior art processes is shown below in Table 1.

In an embodiment of the present disclosure, there is provided a solid-solid flow reactor used to synthesize the compound of formula (I). The flow reactor consists of the parts of: (1) solid dosing-1 and (2) solid dosing-2 to add the solid reactant. The (3) motor is used to rotate the (6) screw conveyor in the (5) Jacket. The jacket includes (4) jacket outlet, (7) jacket inlet and outlet to obtain the (8) product.

Although the subject matter has been described with reference to specific embodiments, this description is not meant to be construed in a limiting sense. Various modifications of the disclosed embodiments, as well as alternate embodiments of the subject matter, will become apparent to persons skilled in the art upon reference to the description of the subject matter. It is therefore contemplated that such modifications can be made without departing from the spirit or scope of the present subject matter as defined.

### EXAMPLES

The disclosure will now be illustrated with the working examples, which is intended to illustrate the working of disclosure and not intended to take restrictively to imply any limitations on the scope of the present disclosure. Unless defined otherwise, all technical and scientific terms used herein have the same meaning as commonly understood to one ordinary person skilled in the art to which this disclosure belongs. Although methods and materials similar or equivalent to those described herein can be used in the practice of the disclosed methods and compositions, the exemplary methods, devices, and materials are described herein. It is to be understood that this disclosure is not limited to particular methods, and experimental conditions described, as such methods and conditions may apply. Following examples are given by way of illustration and therefore should not be constructed to limit the scope of the disclosure.

### Example 1

### Synthesis of Compound of Formula (3)

A solution of methyl magnesium bromide (2 mmol) was added into indole (1 mmol) in 50 ml of dichloromethane, drop wise over 45 min under argon at 0° C. After stirring for 30 minutes, the solution was cooled to −20° C. and then adding ethyl epoxy butanoate (1 mmol) in dichloromethane for 1.5 hour at −20° C. After stirring for 1 h, IN HCl was added. The organic layer was separated, and the aqueous layer was extracted with 3-time dichloromethane. The combined organic layer was dried over anhydrous MgSO₄ and concentrated in vacuum to afford compound of formula (3).

¹H NMR ¹H NMR (400 MHZ, CHLOROFORM-d) d=7.69 (d, J=7.9 Hz, 1H), 7.35 (d, J=8.0 Hz, 1H), 7.25-7.12 (m, 2H), 7.09 (d, J=2.1 Hz, 1H), 4.27 (q, J=7.1 Hz, 2H), 3.74-3.52 (m, 1H), 1.36 (d, J=7.1 Hz, 3H), 1.32-1.27 (m, 3H). 13NMR ¹³C NMR (101 MHz, CHLOROFORM-d) d=174.3, 136.1, 126.5, 122.0, 121.9, 119.3, 118.7, 117.3, 111.2, 77.3, 76.7, 74.1, 61.7, 34.6, 14.1.

### Example 2

### Synthesis of Compound of Formula (I)

Compound of formula (3) (1 mmol), tertiary butoxide (2 mmol) and N methylated quinidine (4) (1 mmol) was passed through in the screw feeder reactor rotating with 80-120 rpm at 25-30° C. After the residence time of 30-60 seconds, the light yellowish solid obtained was analyzed by TLC, which was marked with the completion of the reaction. The reaction mixture was filtered using a sintered funnel. Washed the reaction mixture with EtOAc and evaporated under reduced pressure. The crude product was purified by flash column chromatography to get the compound of formula (I), indolmycin.

Indolmycin ¹H-NMR (DMSO-d₆) 10.93 (1H, br s), 8.60 (1H, br s), 8.31 (1H, s), 7.48 (1H, d, J 57.8 Hz), 7.37 (1H, d, J 57.8 Hz), 6.85-7.15 (3H, m), 4.6 (1H, d, J 52.4 Hz), 3.89 (1H, m), 2.71-2.83 (3H, m), 1.23 (3H, d, J 57.0 Hz), d: 1.19.

Various substituted indole reacting with methyl magnesium bromide in a chloroform at −20° C., followed by adding ethyl epoxy butanone in DCM at −20° C. for a period of 1.0 hr and maintaining reaction mixture at −20° C. for 1.0 hr to afford the compound substituted ethyl 2-hydroxy-3-(1H-indole-3-yl) butanoate.

Further, passing the substituted ethyl 2-hydroxy-3-(1H-indole-3-yl) butanoate obtained by above step were reacted with tertiary butoxide and N-methylated quinidine through solid-solid continuous flow reactor for 1.0 minute to obtain different substituted Indolmycin derivatives (R).

### Example 3—Comparative Study

The comparative study of the present disclosure with prior art processes indicates that the process of the present disclosure is highly economical for commercialization giving high yield of at least 70% with less steps and lesser time. Comparative study with prior art processes is shown below in Table 1.

TABLE 1

| | Present | Previous | Corresponding |
|---|---|---|---|
| | disclosure | synthesis | prior art |
| Total | 2 steps | 3 to 4 | WO9952095 |
| Reaction | | steps | |
| step | | | |
| Total | 4-5 hours | 6-7 days | J. Org. Chem. 1986, 51, |
| Reaction | | | 4920-4924 |
| Time | | | |
| Overall | 72%-90% | 50%-70% | J. Org. Chem. 1986, 51, |
| yield | | | 4920-4924, chem. Pharm. |
| | | | Bull, 38 (2) 323-328 |
| | | | (1990), Tetrahedron |
| | | | Letters, Vol. 37, No. 36, |
| | | | pp. 6447-6448, 1996 |
| Solvent | No solvent used | Required in | |
| | in second step | each step | |
| Operation | In continuous | Batch | — |
| | flow | | |

### ADVANTAGES OF THE DISCLOSURE

The present disclosure provides an extremely simple and cost-effective process for the preparation of Indolmycin and its derivatives is provided. The new process disclosed herein, unlike the prior arts in the field, involves only two steps. The process disclosed herein is only of 4-5 hrs. compared to prior lengthy processes of 6-7 days for the preparation of Indolmycin. The present disclosure employs a continuous flow reaction for gram scale synthesis. The limited usage of solvents makes the process disclosed in the present disclosure advantageous in terms of commercial aspect.

We claim:

1. A continuous flow process for the preparation of Indolmycin and its derivatives compound of formula (I), 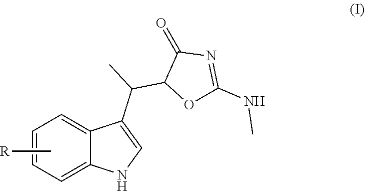
wherein,
R is selected from the group comprising of hydrogen, C₁-C₁₀ alkyl, C₁-C₁₀ aryl, alkyl aryl, haloalkyl, alkyl hydroxy, alkoxy, hydroxy, halo, cyano, heteroaryl, heteroalkyl, alkyl heteroaryl, substituted or unsubstituted C₁-C₁₀ aryl/alkyl, C₂-C₁₀ alkenyl, alkenyl aryl, alkenyl heteroaryl, C₂-C₁₀ alkynyl, alkynyl aryl, alkynyl heteroaryl, C₃-C₁₀ cycloalkyl, heterocyclic alkyl, alkyl cycloalkyl, alkyl heterocycloalkyl, alkyl carboxy, acyl, alkyl acyl, alkyl acyloxy, alkyl alkoxy, alkyl carbonyl, aryl carbonyl, alkoxycarbonyl, alkyl alkoxycarbonyl, aminocarbonyl, alkyl aminocarbonyl, alkyl acylamino, alkyl ureido, amino, alkyl amino, sulfonyloxy, alkyl sulfonyloxy, sulfonyl, alkyl sulfonyl, sulfinyl, alkyl sulfinyl, alkyl sulfanyl and alkyl sulfonyl amino;
wherein said process comprising the step of:
a) reacting indole derivative compound (2) 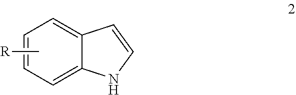
with methyl magnesium bromide in a suitable solvent at a temperature in the range of 0-30° C., followed by adding ethyl epoxy butanone compound (1) 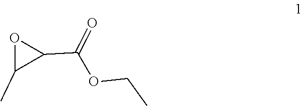
in a suitable solvent at −20° C. for a period of 1-2 hrs and maintaining the obtained reaction mixture for 1 hr at −20° C. to obtain the compound of formula (3); and 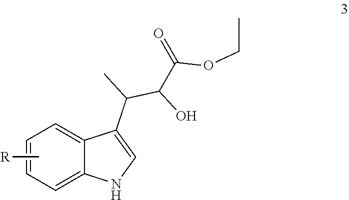
b) passing the compound of formula (3) obtained at step a), base and N-methylated quinidine (4) through solid-solid continuous flow reactor for 1 minute to obtain the compound of formula (I).

2. The continuous flow process as claimed in claim 1, wherein the R is hydrogen or C₁-C₁₀ alkyl.

3. The continuous flow process as claimed in claim 1, wherein the R is hydrogen.

4. The continuous flow process as claimed in claim 1, wherein the solvent is selected from tert-butyl alcohol, dichloromethane, ethyl acetate and water, methanol, ethanol, acetone, methyl ethyl ketone, acetonitrile, propionitrile, formamide, N,N-dimethylformamide, dioxane, tetrahydrofuran, dimethylsulfoxide, sulfolane, hexamethylphosphoramide and combination thereof.

5. The continuous flow process as claimed in claim 1, wherein the base is selected from tertiary butoxide, sodium tert butoxide, triethylamine, diisopropylethylamine, pyridine, lutidine, procaine, 2-phenyl ethyl benzylamine, dibenzylethylenediamine, ethanolamine, diethanolamine, tris hydroxymethyl aminomethane, polyhydroxy alkylamine, or N-methyl glucosamine.

6. The continuous flow process as claimed in claim 1, wherein the reaction temperature is in the range of −20° C. to 20° C.

7. The continuous flow process as claimed in claim 1, wherein the reaction time is in a range of 2-5 hours.

8. The continuous flow process as claimed in claim 1, wherein yield of the compound of formula (I) is at least 70%.
